(19) United States
(12) Patent Application Publication
(10) Pub. No.: US 2024/0401123 A1
(43) Pub. Date: Dec. 5, 2024

(54) METHOD FOR DETECTING RARE MUTATIONS IN LIQUID BIOPSY

(71) Applicant: FLOATING GENES, EVRY-COURCOURONNES CEDEX (FR)

(72) Inventor: Dipanwita BISWAS, IVRY-SUR-SEINE (FR)

(73) Assignee: FLOATING GENES, EVRY-COURCOURONNES CEDEX (FR)

(21) Appl. No.: 18/697,347

(22) PCT Filed: Sep. 30, 2022
(86) PCT No.: PCT/FR2022/051851

§ 371 Date: Mar. 29, 2024

(30) Foreign Application Priority Data
Sep. 30, 2021 (FR) FR2110373

(51) Int. Cl. C12Q 1/6858; C12Q 1/6827

(52) U.S. Cl. CPC C12Q 1/6858; C12Q 1/6827

(57) ABSTRACT

A method of detecting rare mutations in a biological sample. More specifically, a method for detecting rare mutations involving a first step of amplifying mutations by PCR, a second step of labelling sequences containing a uniquely amplified mutation with a Unique Molecular Identifier (UMI), and a third step of correcting sequencing errors using the UMI. This method can be applied in many medical fields, such as the detection of cancer mutations at a very early stage, the detection of infectious diseases, the detection of immune diseases as well as inflammatory diseases, the screening of antibiotic resistance, prenatal diagnosis, the detection of mitochondrial DNA mutations, etc.

The present invention relates to the field of detection of rare mutations in a biological sample. More precisely, it concerns a method of detection of rare mutations involving a first step of mutation enhancement by PCR, a second step of barcoding the unique amplified sequences containing a mutation with a unique molecular identifier, and a third step of correction of sequencing error using barcoding to resolve the errors. This method can be applied in many medical fields such as detection of cancer mutations at a very early stage, detection of infectious disease, detection of immune disease as well as inflammatory diseases, screening of antimicrobial resistance, prenatal diagnosis, detection of mitochondrial DNA mutations etc.

### BACKGROUND OF THE INVENTION

Apoptotic cells, including tumor tissue, release their nucleic acids into the peripheral bloodstream and are therefore readily accessible from body fluids such as blood, urine, etc. (Stroun et al. 1987). The analysis of nucleic acids, proteins or metabolites derived from body fluids is called “liquid biopsy”. Compared with tissue biopsy derived from a single tumor site, the main advantage of liquid biopsy is its easy and non-invasive accessibility, repeated sampling capability and broad coverage of tumor heterogeneity (Ilie and Hofman 2016; Gonzalez-Billalabeitia et al. 2019).

Circulating free DNA has recently emerged as a promising biomarker and has potential to revolutionise detection and monitoring of cancer: diagnosis, monitoring clonal evolution, resistance and drug response (Abbosh et al. 2017; Qin et al. 2016; Tie et al. 2015 et 2016). Circulating tumor-derived DNA (ctDNA) can provide a broader picture of a cancer patient's mutational profile than localized tissue biopsy. The mutational profile includes qualitative (nature of mutations) as well as quantitative (relative ratio of each mutation present) characterization of genes, and this patient-specific profile aids in the personalization of treatment and disease monitoring. Detection of circulating tumor DNA (ctDNA) in the vast background of cell free DNA released from healthy cells is challenging due to their rare occurrence (Schwarzenbach et al. 2008; Forshew et al. 2012; Kennedy et al. 2014). Often these numbers are too low to be represented in percentage but are countable in absolute numbers. For example, in the early stage of cancer, a 2-3 cm tumor can release as low as 10 ctDNA per 10 ml of blood (Fiala et Diamandis 2018).

A whole range of ctDNA detection technology exists in the market. The sequencing-based cancer detection technologies can be divided into two main categories: 1) non-targeted sequencing 2) targeted detection. The first approach includes whole genome or whole exome sequencing to find out unknown point mutations or the copy number variations (CNV). The advantage of non-targeted sequencing is its ability to identify novel changes occurred during the tumor evolution without prior knowledge of the tumor genes. Despite this advantage, a high concentration of cfDNA input is necessary to have reliable mutation detection and overall sensitivity of these non-targeted sequencing techniques remains low (can detect mutation above the level of 5-10%) due to low depth of data. In a shallow or low pass whole genome sequencing, each locus is read once, which is not sufficient to give enough confidence to the data.

On the other hand, the targeted sequencing approach is highly sensitive, and mutations can be detected in the allele frequency rate of 0.01% in a fast and cost-effective manner. The quality of the analysis is superior to those obtained by non-targeted methods due to higher depth of the sequencing data. The major disadvantage of targeted sequencing is that it requires detailed information on tumor genetics. Several targeted sequencing technologies have been invented in the past decades. In the “deep sequencing” approach, a target region is sequenced with a very high coverage (˜10,000×). The advantage of deep sequencing is to characterize multiple biomarkers in parallel while its disadvantage being extreme high depth needed to detect low allelic frequency and therefore drastically increasing sequencing costs. Apart from high depth deep sequencing other sequencing technologies are based either on target enrichment (TAm-seq, Capp-seq) or generating high quality data with relatively fewer reads by tagging unique molecular identifiers (UMI) to each DNA sequence in order to correct sequencing errors (Safe-seq, Duplex-seq). For the first time, target enrichment method of Capp-Seq (Newman et al., 2014) together was integrated with duplex-seq (Schmitt et al. 2012) double stranded UMI for error correction to make Capp-seq/iDES (Diehn et al, patent WO 2016/040901) that reached the mutation detection limit of 0.0025%. TAm-seq (Gale et al. 2018) and Capp-Seq are targeted sequencing approaches, and these technologies enrich the target regions indifferent of wildtype or mutant sequences. Therefore, the ratio between wild-type and mutant remains same before and after the enrichment process. This does not solve the problem of early detection where the amount of ctDNA is extremely low and wild-type DNA might be 1000-fold in excess. All the previously described technologies can help little for the early detection of cancer or tracing minimal residual disease where the signal of ctDNA can be masked by the cfDNA coming from the healthy tissues. A PCR technique called “COLD-PCR” (Li et al. 2008 et 2009) and its variants “ice COLD-PCR (Milbury et al. 2011) or e-iceCOLD PCR” (How-Kit et al. 2013) can enrich minority mutant alleles as compared to the wild-type and therefore change the ratio between wild-type and mutant. However, the COLD PCR technique is not efficient when more than one gene needs to be detected in tandem. A new primer construction strategy had been proposed by Huang et al. (BMC Biotechnology, 2019, 19:62) where the special primers called ‘stuntmers’ can preferentially augment the mutants as compared to the wild-type counterparts. One stuntmer can detect several tandem mutations and can significantly raise the mutant alleles during the PCR step therefore changing the ratio between the mutant and wildtype. Stuntmers can be a promising tool for early-stage cancer detection and can be coupled with high-throughput sequencing technologies.

In prior art methodology, mutant allele enrichment method like COLD-PCR, and other correction methods for sequencing by tagging have been validated individually, as summarized in Table 1. Only Capp-Seq/IDES combines enrichment of mutant and correction of sequencing errors.

TABLE 1
Different sequencing technologies with target
enrichment or error correction options.

| | Amplification | Enhancement of | Enrichment |
|---|---|---|---|
| | of total | mutant targets | by probe |
| | targets by PCR | by PCR | hybridization |
| Target | TAm-seq | COLD-PCR | Capp-seq |
| enrichment | (Gale et al. 2018) | (Li et al. 2008, 2009) | (Newman et al. 2014) |
| | | ice COLD-PCR | |
| | | (Milbury et al. 2011) | |
| Barcoding | Safe-seq | | |
| | (Kinde et al. 2021) | | |
| | Duplex-seq | | |
| | (Schmitt et al. 2012) | | |
| Target enrichment | | “Enhance-seq” | Capp-seq + iDES |
| and Barcoding | | | (Diehn et al, patent |
| | | | WO 2016/040901 A1) |

Next generation sequencing (NGS) technique is the best way to make a molecular genetic profile related to cancer genes as it can provide both qualitative and quantitative results. NGS is based on several millions of short DNA pieces sequencing in parallel (Glenn 2011) followed by de novo assembly or alignment to a reference genome. NGS shows a relatively high error rate (0.1 to 1%) depending on the type of the platform used and therefore makes the rare mutation detection highly challenging. To overcome this drawback, several technologies have been proposed to improve mutation detection in ctDNA. Following Table 2 presents an overview of the existing technologies to improve the ctDNA detection.

TABLE 2
Summary of different sequencing technologies

| | Enrichment of | Sequencing error | Limit of | |
|---|---|---|---|---|
| NGS platform | target | correction | detection | Reference |
| NGS Deep Seq | No | No | 0.02% | Narayanan et al. 2012 |
| (10,000x) | | | | Couraud et al. 2014 |
| | | | | Uchida et al. 2015 |
| Safe-seqS | No | Yes | 0.1% | Kinde et al. 2011 |
| | | | | Tie et al 2015, 2016 |
| | | | | Fredebohm et al. 2016 |
| Duplex-seq | No | Yes | 0.01% | Schmitt et al. 2012 |
| TAm-Seq | Yes | No | 0.02% | Gale et al. 2018 |
| CAPP-seq | yes | No | 0.02% | Newman et al. 2014 |
| CAPP-seq/iDES | yes | Yes | 0.0025% | Newman et al. 2016 |

The drawback of NGS based high throughput analysis is that it lacks sensitivity due to its high error rates during sequencing process. The challenge of detection of rare mutations in the circulating free DNA is therefore mainly due to two factors: (i) very low amount of circulating tumor DNA in the early-stage cancer and (ii) the high error rate of the NGS machine. There is thus a need for more sensitive and high-throughput methods to detect and monitor tumor-derived nucleic acids in cancer patients.

### SUMMARY OF THE INVENTION

The inventor made a pipeline called “Enhance-seq” technology to generate high-fidelity sequencing data by combining mutation enrichment with stuntmer inspired primers, called Enhancers, followed by unique molecular identifier (UMI) based error correction methodology. More precisely, the method of the invention is a two-steps pipeline for rare mutation detection: the first stage is a mutation-enriching PCR based on the use of Enhancer sequences; these Enhancer sequences specifically increase the number of mutated copies present in the population, while at the same time blocking amplification of the wild-type sequence. In the second step the “Corrector” molecules containing unique molecular identifier (UMI) are joined to each DNA molecule to correct the errors of the high-throughput next generation sequencing.

The method of the invention allows the detection of a rare mutation in a biological sample, and includes the steps of:
Step 1: Providing a sample comprising DNA
Step 2: Amplifying selectively the region containing said mutation using Mutation Enhancement PCR (MEP) technology, which includes the following steps:
a) Providing a PCR primer (Enhancer) comprising 3 regions:
   I. a region called “R-region”, which specifically binds to the wild-type sequence, but cannot bind if there is a mutation
   II. a linker and
   III. a region called “E-region”, which specifically binds to a sequence located ahead of the mutation site;
b) Amplification of the template DNA by a PCR comprising two hybridization steps: (i) a first hybridization at a temperature T1 which allows binding of the R-region of the primer to the wild-type sequence and (ii) a second hybridization at a temperature T2 which allows binding of the E-region of the primer; wherein T1 is at least 5° C. higher than T2;
c) In case of binding of the E-region, the sequence containing the said mutation is amplified
d) Obtaining amplicons containing the said mutation. The said amplicons containing an oriented restriction enzyme site, the said oriented restriction enzyme site being added either during the MEP, or during an additional PCR after step c) of the said MEP, using primers containing an oriented restriction enzyme site.
   Step 3: Adding a Corrector sequence at each end of the resulting amplicon by PCR, comprising a fixed sequence, a unique molecular identifier (UMI), an oriented restriction enzyme site compatible with the oriented restriction enzyme sites presents in the extremities of the said amplicons, and optionally a barcode specific to the sample/patient, by using the said restriction sites
   Step 4: Amplifying amplicons resulting from Step 3 by PCR
   Step 5: Sequencing amplicons resulting from Step 4 by NGS sequencing.
   Step 6: Correcting sequencing errors by bioinformatic analysis using the unique molecular identifiers (UMI).

The invention also concerns an amplifying primer comprising a restriction site in the E-region.

### Advantages of the Invention

The method of detection of rare mutations of the invention provides advantages over prior art for several reasons. First, this method relies on the ability to selectively amplify rare mutations using mutation enhancement technology involving the use of Enhancer. PCR with Enhancers provides a simple, highly sensitive and accurate way of rare mutation detection. The Enhancers (also called stuntmers) are designed so that when they are used as forward primer in a PCR reaction, they can suppress the amplification of the wildtype template and preferentially permit the amplification of the mutant forms. Design of Enhancers are based on wild-type sequence and does not depend on mutation therefore the same Enhancer can identify several tandem mutations in a hotspot site including point mutation, insertion, deletion and rearrangements. Blocking and amplification occurs with the same primers. As the mutations are amplified, it is possible to find back the rare mutations even after the downstream processing that involves loss of DNA in the pipeline. This step allows at least 5-fold increase of rare mutations frequency in the sample.

The unidirectional binding of Corrector sequences enables targeted and efficient amplification of mutated sequences, with the result that the sequencing depth required is much lower than with state-of-the-art methods (around 3 times fewer sequences to read). This advantage is illustrated in FIGS. 4, 5 and 6, in comparison with alternative mutation amplification methods.

FIG. 4 illustrates a method in which UMIs are added by PCR. Each DNA molecule is amplified several times with different UMIs, leading to the need to analyze a large number of sequences due to the presence of several clusters for the same molecule. This method requires high sequencing depth.

FIG. 5 illustrates a method in which UMIs are added by homopolymeric linkage (TA cloning). TA cloning involves several steps to be carried out in parallel or in succession, for example: 1) T-tail, 2) A-tail, 3) Ligation. This makes the protocol longer and more elaborate and adds a single base to the adaptor/amplicon ends by successive linkages. As the binding reaction relies on a single base pair, it cannot ensure that 100% of the amplicons containing the mutation are actually bound, which can easily lead to DNA molecules containing rare mutations subsequently being missed. The method is therefore unreliable, as some mutations may not be lost in the process.

FIG. 6 illustrates the method according to the invention based on the addition of UMI using the Golden gate method. The binding of each amplicon to the adapter is efficient and directed thanks to the presence of a cohesive end formed by 4 bases with a different pattern at each end. Each sequence is then bound to a Corrector adapter for amplification, and only one copy per sequence is prepared. Sequences can be read in both sense and antisense, if greater precision is required. This method ensures that all mutated sequences are amplified, thus enabling a reliable diagnosis.

Secondly, the method relies on the use of a unique molecular identifiers (UMI) that permit to correct the sequencing error after NGS reading. This UMI is introduced through forward and reverse adapter molecules that have been designed with a Unique Molecular Identifier (UMI) on both sides of the adapter molecule.

The labelling step is innovative in that it relies on a Golden Gate-based UMI labelling approach to correct sequencing errors (see above). For the first time, the inventor proposes a labelling approach based on Golden gate technology. In the known Golden gate method, binding is directional. Here, the innovative approach consists in the sense and antisense primers of the adaptor molecule (here Corrector sequence) having a directionality to bind specifically with insertion molecules, and the design consists of a patient-specific label followed by a unique molecular identifier (UMI) on both sides of the adaptor molecule. Directionality is conferred by the presence of the restriction site added to the ends of the amplicon. The UMI is used to correct sequencing errors after NGS reading.

With respect to the other barcoding technologies (Duplex-seq) which are based on ‘A’ tailing followed by ligation, Golden gate based barcoding technology is more robust because 4 base-pair overhang permits to have a stronger ligation efficiency and different overhangs at the two ends permits ligation specificity. In contrast to the other barcoding technologies (Duplex-seq) that are achieved in two or more steps, this barcoding technology set up by the inventors is done in only one step therefore is more practical.

Thus, this method relies on the enrichment of mutations using a first PCR (Mutation Enhancement PCR) followed by barcoding (see FIG. 7). The step of barcoding allows to tag each sequence before amplification for sequencing so that it becomes possible to distinguish two different DNA molecules which are the product of the same PCR and to statistically analyze the result of sequencing in order to correct sequencing error through bioinformatic analysis. The use of barcoding permits to discard any false positive and thus to significantly increase the sensitivity of the method (lowers the limit of detection).

Another advantage of this technology is that it does not require very deep sequencing, as detection is highly sensitive thanks to the selective and reliable enrichment of mutations (all mutations, even very rare ones) prior to the amplicon amplification stage. In addition to enabling the detection of very small quantities of mutations, the increased sensitivity means that the result can be read using a new-generation, low-depth sequencing machine (e.g. Illumina iSeq 100), since the signal-to-noise ratio for mutants is high, whereas with prior art methods, the detection of rare mutations requires expensive machines. Indeed, “deep sequencing” corresponds to 10,000 reads/target. With the method of the present invention, a maximum of 2,000 reads/target is required, but good read quality can be achieved with 1,000 reads/target, and even with 500, 100 and as low as 50 reads/target. The lower number of reads required for good data quality will enable users to switch from very expensive high-end sequencers (around 850,000 to 1 million euros) to mini-sequencers (i-seq100) around 20 times cheaper (around 20,000 euros). As a result, high-quality data can be recovered from shallow sequencing with little capital investment. As a result, the use of this technology will lower the cost of sequencing.

The method advantageously includes the use of sample- or patient-specific labelling so that different patient samples can be pooled and processed simultaneously in the NGS sequencing step. The method is easy to implement, as it is based on PCR and NGS, which are routine technologies. It can be offered as a kit tailored to a particular diagnosis, which is very convenient for users.

The “Enhance-seq” method has advantages over COLD PCR because Enhancers can detect mutations in tandem, as shown by Huang et al. 2019. “Full Cold PCR” cannot work with low DNA concentration and requires a substantial accumulation of PCR products for mutation enrichment to occur; enrichment therefore only occurs in the last PCR cycles. The inventor's experimental results show that Enhance-seq can operate with extremely low DNA concentration and exhibits 10- to 500-fold enrichment (Tables 3 and 5) depending on the initial template.

Experimental results confirm that “Enhance-seq” technology will enable diseases to be diagnosed earlier than other technologies in the state of the art, thanks to more sensitive detection of rare mutations present in very low numbers in the early stages of these diseases. As it can detect very small quantities of mutated DNA in a complex sample such as a liquid biopsy (e.g. from blood, urine, cerebrospinal fluid, tears, etc.), the method is particularly useful in the field of oncology for screening, detection of risk factors, early diagnosis, prognosis, treatment personalization and for monitoring treatment efficacy.

Thanks to its high sensitivity, the method can be performed using only a small sample volume, particularly a blood sample. It may be particularly suitable for diagnosing vulnerable patients, such as children, whose state of health does not allow several ml of blood to be taken.

### DETAILED DESCRIPTION OF THE INVENTION

A first object of the invention is a method for the detection of a rare mutation in a biological sample, comprising the following steps—the detection of a rare mutation in a biological sample.
Step 1: Providing a sample comprising DNA.
Step 2: Amplifying selectively the region containing said mutation using Mutation Enhancement PCR (MEP) technology, which includes the following steps:
a) Providing a PCR primer (Enhancer) comprising 3 regions:
   I. a region called “R-region”, which specifically binds to the wildtype sequence, but cannot bind if there is a mutation
   II. a linker and
   III. a region called “E-region”, which specifically binds to a sequence located ahead of the mutation site;
b) Amplification of the template DNA by a PCR comprising two hybridization steps: (i) a first hybridization at a temperature T1 which allows binding of the R-region of the primer to the wild-type sequence and (ii) a second hybridization at a temperature T2 which allows binding of the E-region of the primer; wherein T1 is at least 5° C. higher than T2;
c) In case of binding of the E-region, the sequence containing the said mutation is amplified
d) Obtaining amplicons containing the said mutation. The said amplicons containing an oriented restriction enzyme site, the said oriented restriction enzyme site being added either during the MEP, or during an additional PCR after step c) of the said MEP, using primers containing an oriented restriction enzyme site.
Step 3: Adding Corrector sequence at each end of the resulting amplicon by PCR, comprising a fixed sequence, a unique molecular identifier (UMI), an oriented restriction enzyme site compatible with the oriented restriction enzyme sites presents in the extremities of the said amplicons, and optionally a barcode specific to the sample/patient, by using the said restriction sites.
Step 4: Amplifying amplicons resulting from Step 3 by PCR
Step 5: Sequencing amplicons resulting from step 4 by NGS sequencing.
Step 6: Correcting sequencing errors by bioinformatic analysis using the unique molecular identifiers.

As used herein, “rare mutation” means any genetic modification of DNA, or RNA (for example mRNA) that is a marker of a disease or a pathological state, or of a predisposition to develop a disease or a pathological state, or more generally a marker of the presence of a rare DNA molecule in a complex sample. It can correspond to a point mutation, a deletion, an insertion, or a rearrangement. In its broadest sense as used in the scope of the invention, the term “mutation” corresponds to a rare genetic event compared to a reference sequence. The reference sequence may for example be the DNA sequence of a transplant receiver compared to the graft sequence, a first generation of a SARS-Cov2 virus compared to an emerging variant of the virus. It is understood that the method for detecting rare mutations according to the invention allows the detection of one or more mutations simultaneously, notably under multiplexing conditions.

A Rare mutation can also result from methylation affecting the expression of DNA, especially methylation occurring in regulatory sequences such as promoter or Enhancer regions. In this case, the rare mutation corresponds to the presence of a cytosine as a marker of DNA methylation and the method thus comprises a step of treatment of the DNA sample before DNA amplification of Step 2 to reveal the methylation site. This step can be performed using method known from a skilled person in the art such as bisulfide conversion or enzymatic conversion.

A rare mutation can correspond to SNP, or more generally to a variant sequence. Further, a rare mutation can affect the RNA, including without limitation mRNA, but also possibly siRNA, miRNA; in this case, it can be detected by the method of the invention after conversion of mRNA into cDNA using reverse transcriptase. Accordingly, the method comprises a step of treatment of the DNA sample before DNA amplification of Step 2 in order to convert RNA into DNA.

A mutation is considered as “rare” when it is represented in less than 50% of the DNA of the analyzed sample. Preferably, the method of the invention is applied to the detection of mutations having a frequency of 1% or less, especially a frequency of less than 0.1%, or less than 0.001% or less than 0,0001%. The limit of detection of the method has been evaluated around 0,00001% or less; this range of detection limits is compatible with the detection of ultra-rare mutations. The method detected 10 mutant DNA molecules in a context of 10\hat{ }9 wild-type molecules.

Step 1 of the method consists in providing a sample containing DNA. This sample is a biological sample or is derived from a biological sample.

As used herein, “biological sample” means a sample comprising at least DNA or RNA. It can be a complex biological fluid such as blood, urine, tears or an extract of such fluid which contains DNA or RNA. The biological sample can also be prepared from a prokaryotic or eukaryotic cell, virus or any other biological tissue.

The method of the invention is particularly relevant for the detection of rare mutations in free circulating DNA (cfDNA) in a liquid biopsy.

Step 2 of the method consists in a selective amplification of rare mutations through Mutant Enhancement PCR (MEP) derived from work of Huang et al, 2019. The MEP technology relies on the use of an “Enhancer primer”.

As used here, an “Enhancer” is a so-called “Enhancer primer” containing three regions arranged from the 5′ end to the 3′ end in the following order—the recognition region (region-R), the linker region and the extension region (region-E) (FIG. 1). The region-R is designed to recognize a mutation-rich site (hot spot) and hybridize to the wild-type version of the sequence. In the event of mutation, binding of the R-region is compromised. The E-region binds to sequences upstream of the hotspot. The Tm (hybridization temperature) of the R-region is around 5 to 10° C. higher than that of the E-region. Approximately 4 to 10 bps of the 3′ end sequence of the E-region overlap the 5′ end of the R-region. The linker region can be composed, for example, of a spacer region comprising from 3 carbons (“C3 spacer”) to 12 carbons (“C12 spacer”), for example 3, 6, 8, 9 or 12 carbons. This spacer region is a non-nucleotide region capable of blocking sequence amplification.

In a first particular embodiment, the Enhancer consists of 3 regions with the presence of a restriction site oriented upstream of the E-region. The cohesive ends produced at the 5′ and 3′ ends of the amplicon are different. This difference enables specific binding at each end. See FIGS. 2 and 3. PCR with Enhancer (MEP) has two hybridization temperatures to facilitate the binding of two primer regions alternately. The binding of the template to the R or E region of the Enhancer primer depends on the sequence of the template, whether wild-type or mutant, and is controlled by the hybridization temperatures. The R-region of the Enhancer is designed to bind to the wild-type version of the template in the hot-spot region. During the first hybridization, all wild-type sequences are saturated by the binding of the activator to the R-region. In this binding configuration, PCR cannot proceed. In this binding configuration, PCR cannot proceed due to the presence of a non-nucleotide structure in the linker region, so no product is formed. On the other hand, if a mutant version is present in the template in the hotspot region, binding to the R-region is compromised due to base-pair mismatch. In this case, the E region enables binding to the mutant version. The second hybridization temperature enables the E-region to bind to the upstream hotspot region, possibly forming a product. PCR with mutation enrichment (MEP) results in blocking of the wild-type version and amplification of the mutated version.

When the E-region includes an oriented restriction site sequence, a restriction site is directly integrated at each end of the amplicon obtained in step 2. Such a construction reduces the time and cost of the method. An illustration of this construction and its implementation can be found in FIGS. 2, 3 and 7.

The oriented restriction site can be a site recognized by a type II or type III restriction enzyme; however, it is preferred to use a type II restriction enzyme site as type II restriction enzymes allow directional insertion with greater flexibility than type III restriction enzymes. A restriction site usable in the method according to the invention may be the site of the Bsal restriction enzyme, but other enzymes are also such as BbsI, BsmBI, etc.

In an even more particular embodiment of the invention, the 5′ end and the 3′ end of the adapter linker region are designed with two distinct cohesive sequences originating from the Bsal restriction site. These separate binding sequences add specificity to each end. Binding is preceded by restriction digestion with Bsal. The 4-base cohesive end in Golden Gate technology enables greater binding efficiency than the single-nucleotide cohesive end used in TA cloning. As a result, a double-stranded consensus sequence is present in both the 5′ and 3′ directions of the amplicon, increasing the reliability of the sequencing data.

When a restriction site is added to the E-region of the primer, it may be advantageous to introduce LNA bases into the R-region of the primer. This addition makes it possible to increase the Tm of the first hybridization (T1) so that the difference between T1 and T2 is greater than 5° C., making it possible to better discriminate between the two conditions and specifically amplify sequences carrying a mutation.

As used here, “LNA bases” are modified nucleic acids (locked nucleic acids). These are a class of high-affinity RNA analogues in which the ribose ring is “locked” in the ideal conformation for Watson-Crick binding. This structure gives LNA bases enhanced thermal stability and enables stable hybridization with shorter hybridization sequences.

LNA bases can also be used in other ways, as described below, and these methods can be combined with one another.

In a second particular embodiment, a restriction site is added by performing an additional PCR, after step c. of the mutation enrichment PCR, using primers containing an oriented restriction site. At the end of this step, the amplicon obtained is flanked by the sequence of a restriction site at each of its ends. The DNA is then digested with the restriction enzyme corresponding to the restriction site sequence, revealing the cohesive ends needed for effective binding to Corrector sequences.

The addition of a restriction site at each end of the amplicon enables integration of the Corrector sequence provided for in step 3.

Sense and antisense Corrector sequences are supplied with the same restriction site. To prepare the ends of the Correctors and amplicons, they are digested, for example with a restriction enzyme such as Bsal. Thanks to the use of restriction sites, the cohesive end produced after digestion is specific to the 5′ and 3′ ends, making binding highly specific. Digestion is followed by a binding reaction with T4 DNA ligase.

In another particular embodiment of the invention, a double stranded DNA-specific nuclease (DSN) is added, either before the mutation enrichment PCR, or after the mutation enrichment PCR. The DSN enzyme cuts perfectly hybridized double-stranded DNA.

When the MEP step is accompanied by the prior addition of DSN, amplification of mutation can be improved by cleavage of wild-type DNA before the amplification. This step may contribute to improved multiplexing capacity due to less interference from wild-type DNA. Elimination of wild-type sequences reduces background noise.

When a DSN is added after the MEP step, wild-type sequences are cleaved and eliminated, which favors amplification of amplicons containing the mutations in step 4. In this particular embodiment of the invention, probes specific to wild-type sequences comprising LNA bases are added after addition of the Corrector sequences, together with a DSN. The method is preferably applied under multiplexing conditions, i.e. several mutations are searched for simultaneously using different primer pairs in a single reaction. This procedure is illustrated in FIG. 10 and is as follows:
Step A: PCR enrichment of mutations from cfDNA
Step B: Corrector sequence fixation
Step C: Addition of probes (which may include LNA bases) for pairing with wild-type sequence. Upon probe binding, a DSN is added to the reaction. The DSN will cut any perfectly hybridized duplex (wild-type sequence) and will not cut any mismatched duplex (mutated sequences).
Step D: Uncleaved sequences are recovered for subsequent amplification.

In another particular embodiment of the invention making it possible to increase the proportion of mutants versus wild sequences, the method comprises the addition of probes specific to the mutations and comprising one or more LNA bases coupled to a marker (for example biotinylated probes) after the amplification of step 2 and the addition of Corrector sequences from step 3.

The use of such probes allows the enrichment of mutants. The method can be illustrated in FIG. 11 and proceeds as follows:
Step A: A PCR enrichment of the mutation(s) is carried out from cfDNA.
Step B: Fixing the Corrector sequence
Step C: Attachment of biotinylated LNA probes specific for each mutation. The mutants hybridize with the probe and are therefore taken up by the magnetic streptavidin beads.

Using a DSN method, Keraite et al. 2020, showed that they could reliably predict the initial mutational allele frequency (MAF). Therefore, combining DSN with mutation enrichment PCR will further improve the yield and performance of the method. Indeed, it is expected that the required NGS sequencing depth will be lower and signal quality will be higher.

Step 3 consists in adding a corrector sequence (Corrector) comprising a fixed sequence, a unique molecular identifier (UMI) (allowing UMI labeling) to each end of the amplicon using a restriction site.

A patient- or sample-specific label can be included in the Corrector sequence.

Such a label is specific to each sample and enables different samples to be grouped together for simultaneous processing by NGS sequencing.

The “fixed sequence” is a particular sequence that is common to all amplicons. The fixed sequence is superimposed on the NGS adapters, enabling the sequence to be attached to the flow cell adapter for NGS sequencing. Fixed sequences ensure uniform amplification of all sequences since the same universal primers are used for all sequences prior to NGS.

Indeed, once the templates are amplified by enhanced PCR, the amplicon is linked with the pool of unique of unique adapters called “Correctors” to analyze the nucleic acids in a sample after the next-generation sequencing step. Each Corrector molecule consists of at least one fixed sequence, a unique molecular identifier (UMI) followed by a restriction site, e.g. for a type II restriction enzyme. Corrector sequences are added on either side of each amplicon molecule; the same model is therefore applied for both sense and antisense Correctors. antisense Correctors.

It should be noted that, in view of current developments in NGS technology, particularly regarding its reliability, it is strongly recommended that sequences be read on both the sense and antisense strands. If, in the future, reading only the sense strand (oriented in the 5′-3′ direction) is sufficient, the strand oriented in the 5′-3′ direction may be amplified preferentially.

In an advantageous embodiment of the invention, additional small barcode sequences are associated with mutations to remove any ambiguity as to whether a mutation is present, in the event of an error in amplification or sequencing of UMIs. These small barcodes can be added either at the time of enrichment PCR, due to the presence of such barcodes in the E-region, or at the same time as said oriented restriction site when the latter is added by PCR, or at step 3 due to its presence in the Corrector sequence. In general, these are nucleotide sequences consisting of 1 to 6 nucleotides, preferably 3 to 5.

Step 4 consists in amplifying the amplicons resulting from step 3 by PCR.

The analysis method of the invention enables sequence errors to be corrected thanks to the presence of a unique molecular identifier. At each end of the nucleic acid, a Corrector sense sequence (5′) and a Corrector antisense sequence (3′) containing a universal molecular identifier are added from a pool of unique adapters using a binding site. The UMI gives a unique identity to each DNA molecule. After addition of the Corrector sequence, all molecules are amplified by PCR with a pair of universal primers; each UMI is therefore replicated several times. Errors are corrected starting with each UMI sequence. If a daughter molecule has 50% or more of the same mutation, it is a true mutation, otherwise it is considered a sequencing error. Each UMI is read in both 5′-3′ and 3′-5′ directions.

Step 5 consists of conventional NGS sequencing.

NGS sequencing is a high-throughput technology that enables many samples to be processed simultaneously. As a result, samples can be pooled after step 4. In order to read the results for each patient/sample, the samples for each patient/sample are labelled with a labelling technology in step 3 (patient label or sample label).

In step 6, sequencing errors are corrected by software analysis using unique molecular identifiers (UMIs). The complete method is illustrated in FIG. 9.

The present detection method makes it possible to detect very small amounts of specific DNA sequences in a background of excess wild-type sequences, enabling early detection (screening, diagnosis), prognosis and monitoring of diseases that can be detected by the appearance of specific genetic sequences. It enables the detection of these variants earlier than other state-of-the-art methods.

Consequently, it can be applied to many fields, including oncology, autoimmune diseases, transplant rejection, graft-versus-host disease (GVHD), screening for infectious variants and detection of mutant pathogens (antibiotic resistance).

A second object of the invention concerns an Enhancer primer that enables the integration of a restriction site into the amplicon during enrichment PCR.

Such an Enhancer primer comprises 3 regions:
i. the R region which recognizes and binds specifically to the wild-type sequence, but cannot bind if the region contains a mutation,
ii. a linker region and
iii. the E region, which binds specifically to a sequence located upstream of the mutation site and includes an oriented restriction site sequence positioned upstream of the E-region.

The choice of the oriented restriction site can be made by the person skilled in the art without difficulty. In one particular embodiment, the restriction site is Bsal.

In another particular embodiment, the amplifying primer comprises LNA bases in the R-region.

In an even more particular embodiment, the Enhancer primer comprises both a restriction site in the E-region and LNA bases in the R-region.

The advantages of using such an amplifying sequence and its particular embodiments are described above.

### Applications

The method of the invention can be applied in numerous fields. Some applications are described below.

### Early Diagnosis of Cancer

A tumour releases its genetic material into the bloodstream in the form of DNA, mRNA, small non-coding RNAs and so on. Recovering information about genetic material from a cancer patient's blood sample, using cell-free nucleic acid, is called “liquid biopsy” and has gained in popularity due to its non-invasiveness, repeatability, and dynamic nature. NGS has revolutionized the field of liquid biopsy, and therefore cancer detection, due to its ability to sequence hundreds of cancer hotspots in parallel and establish a patient-specific profile. This personalized genetic profile reveals the main driving mutations to recommend personalized treatment.

Information obtained from DNA can take two different forms. Epigenetic changes in DNA through methylation and somatic mutations in its DNA-point mutation, insertion, deletion, rearrangement, copy number change, etc. To identify the mutations at the origin of cancer, a mutational profile can be generated by NGS-based analysis. On the other hand, cancer detection by NGS suffers from low sensitivity, mainly due to the presence of very small amounts of circulating tumour DNA (ctDNA) and errors generated by the NGS machine. In stage I cancer, a 2-3 cm tumour typically releases around 10 ctDNA in 10 ml of blood (Fiala and Diamandis. 2018). Conventional NGS-based methods (target amplification by PCR followed by sample preparation for NGS sequencing) are not suitable for reaching this detection threshold. “Enhance-seq” presents a method capable of detecting this small amount of mutations present thanks to its detection limit reaching the unprecedented threshold of 10 mutants present in a background of 10\hat{ }9 wild-type DNA sequences (shown in table. 3 in the results section) obtained in a synthetic experiment.

Altered DNA methylation patterns are another early indication of cancer. DNA methylation occurs mainly at cytosine residues followed by a guanine residue on their 3′ flank and are called cytosine-phospho-guanine dinucleotides (CpG islands) (Schultz et al. 2015). There are known changes in the methylation profile for different cancers, particularly in gene promoters. During bisulfide or enzymatic conversion, unmethylated cytosines are converted to uracil while methylated cytosines remain as they are because they are protected by their methyl groups. Sequencing of free circulating DNA (cfDNA) after the conversion step can give us the methylation profile and cancer risk. Methylated cytosines can be considered mutants. The Enhance-seq method can be adapted to detect DNA methylation. Enhancers can be designed for CpG islets. After the bisulfide or enzymatic cytosine conversion step, DNA can be amplified with Enhancers.

In addition to genomic fingerprinting, understanding the functional biology of cells is essential for clinical decision-making. The messenger RNA (mRNA) released by the tumour can reflect any changes in cell function. RNA splicing, fusions, rearrangements, insertion and deletion mutations can be reflected in mRNA. RNA splicing variants can be biomarkers for prognosis and therapeutics (Nilsson et al, 2016; De Fraipont, 2019). Therefore, mRNA can be reverse transcribed into DNA and Enhancers can be designed to detect any dysfunction.

### Non-Invasive Prenatal Test (NIPT)

Non-invasive detection of genetic abnormalities in the foetus from the pregnant mother's blood when there is a suspicion or risk of genetic disease is another application of “Enhance-seq” technology. The most relevant application of “Enhance-seq” technology is the detection of microdeletions, e.g.—Wolf-Hirschhorn syndrome (4p terminal deletion), Cri du chat syndrome (5p terminal deletion), Langer-Giedion syndrome (8p24 deletion), Jacobsen syndrome (11q terminal deletion), Prader-Willi and Angelman syndromes (15q11.2-q13 deletion), DiGeorge syndrome (22q11.2 deletion) etc. Currently, to detect a microdeletion, doctors prescribe whole genome sequencing (WGS) or whole exome sequencing (WES). In the case of low-pass WGS or WES, the sequencing depth is very shallow, and the data is therefore often unreliable. Consequently, any sequencing error leads to invasive amniocentesis. The panel of Enhance-seq “genetic microdeletions will be able to find known genetic deletions very efficiently and overcome “non-reportable results” when there isn't enough DNA. As the technology can work with very small copies of DNA, the test can be performed before the tenth week of pregnancy, when chemical abortion is possible.

### Screening for Antimicrobial Resistance

Pathogens can acquire genetic variants responsible for the development of drug resistance, even if they are present at low frequency in the initial population. This is a serious problem in hospitals, where nosocomial diseases and septicemia cause death. Detecting these rare variants earlier could prevent their spread through earlier surveillance and therapeutic choices, in both the medical and veterinary markets. The following genes confer antibiotic resistance (in brackets)—
rpoB, rpoC (Myxopyronine), rpsL, rrs, rrl, rpsE (Amikacin, streptomycin, spectinomycin), gyrB (Coumermycin, novobiocin), dfrA (Trimethoprim), fusA (Fusidic acid), 23S rRNA genes (Clarithromycin, erythromycin, tylosin), gyrA, gyrB, parC, parE, grlA (Ciprofloxacin, nalidixic acid, norfloxacin), rpoB (Rifampicin) (Melnyk et. al. 2015).
gyrA and grlA in Ciprofloxacin-resistant Staphylococcus aureus mutants (Ferrero et al. 1995).
Mycobacterium tuberculosis becomes drug-resistant by acquiring mutations at seven loci-rpoB, pncA, katG; mabA (fabG1)-inhA, gyrA, gyrB and rrs (Flandrois et al. 2020).

Unique Enhancers can be designed for each antibiotic resistance gene, thus constituting a panel of antibiotic resistance genes. The “Enhance-seq” method can be followed to establish a diagnosis.

### Infectious Diseases-Variant Screening

The presence of very small quantities of pathogens in body fluids can be detected using high-throughput ‘Enhance-seq’ technology, which will enable the screening of several pathogens in parallel.

Beyond simple pathogen detection, variant sequencing is possible with this method, if the wild-type sequence of hotspot regions is known. For example, the receptor binding domain (RBD) of the spike protein of Covid-19 variants. This technology will make it possible to find rare mutations in each wild-type hotspot, whatever the mutation. It could therefore be useful for screening emerging Sars-COV-2 mutants. This is relevant for medical and veterinary markets (e.g. H1N1, H5N1 infections).

### Microbiome Screening

Enhance-seq” will be able to detect rare mutant species in our microbiome (skin, intestine, etc.) to facilitate diagnosis, prognosis, or the personalization of treatments.

### Predicting and Monitoring Organ Transplant Rejection

Cell-free DNA is a good biomarker for organ transplant rejection (Khush et al. 2019). In graft rejection, donor-derived circulating free DNA (cfDNA) present in the recipient's blood originates from damaged cells (Martuszewski et al., 2021; Cheng et al. 2020). The graft's circulating free DNA (cfDNA) is released into body fluids in very small quantities and must be detected from the recipient's cfDNA noise to detect early signs of rejection. The level of donor-derived cfDNA increases before any other protein biomarker (creatinine in the case of kidney transplantation). Early detection of the amount of donor-derived cfDNA in the blood or urine of the organ or graft recipient can guide appropriate treatment and prevent premature graft loss.

If graft rejection is detected, the Single Nucleotide Polymorphism (SNP) profile of the donor and recipient can be established. Custom Enhancers can be designed from the donor SNP profile. The Enhance-seq method can be applied to detect the presence of donor DNA at a very early stage of transplantation (e.g. after a few hours).

### Autoimmune Diseases

There is growing evidence that somatic mutations are linked to autoimmune diseases (Savola et al. 2017; Ross, 2014). Some protein-coding genes contain single tandem repeats (STRs) that are highly mutable in both germline and somatic cells. In somatic cells, a mutated protein coding STR can generate a new, potentially immunogenic protein. These mutations correspond to autoimmunity. In systemic lupus erythematosus (SLE) and Sjögren's syndrome (SJ), an 8-base-pair poly-A sequence is mutated somatically to a 7-base-pair mutant protein (Ross, 2014). Savola et al. have discovered new somatic mutations in patients with with rheumatoid arthritis (RA) while studying the effect of somatic mutations in immune cells (T cells). Using “Enhance-seq” technology, somatic mutation-specific Enhancers can be generated to create gene panels for known autoimmune loci. The “Enhance-seq” method will be applied to discover any somatic mutation from cell-free DNA and establish early diagnosis of autoimmune diseases. This will enable personalized treatment of autoimmune diseases.

### Inflammatory Diseases

Circulating DNA (cfDNA) can be used as a biomarker of inflammation. Genetic mutations or changes in DNA methylation are linked to chronic inflammatory bowel disease (IBD). IBD patients are at high risk of developing gastrointestinal cancer due to the accumulation of insertion-deletion mutations (Olafsson et al. 2020). It is therefore necessary to detect this risk at an early stage. The two main subtypes of IBD are Crohn's disease (CD) and ulcerative colitis (UC). It is generally difficult to distinguish between them, and better patient management requires accurate identification (Mirkov et al. 2017). The ‘Enhance-seq’ IBD sequencing panel can enable accurate identification of the disease at an early stage.

### Neurodegenerative Diseases

Genetic components play a role in the complex multifactorial disorder Alzheimer's disease (AD). Early-onset Alzheimer's disease (EOAD) is caused by a few pathogenic somatic genetic variants in the APP, PSEN1 or PSEN2 genes inherited as an autosomal dominant trait in a few families (Nicolas et al. 2018). Enhance-seq's panel of Alzheimer's disease-specific genes will be able to detect the disease at a very early stage.

When a person suffers a traumatic brain injury (TBI) or ischemic brain damage following cardiac arrest or multiple sclerosis, brain-derived circulating DNA (cfDNA) is released, providing evidence of neurodegeneration (Chatterton et al. 2019). Tracing the methylation pattern for brain origin can be very useful in this case. It is possible to design specific Enhancers of the methylation pattern and identify the fraction of DNA that shows neuronal cell identity and in turn indicates disease severity. See FIG. 8.

### Detection of Mitochondrial Mutation

Mitochondrial DNA mutation is associated with age-related diseases and age-related macular degeneration (AMD). Atilano et al (2021) performed ultra-deep sequencing to differentiate very low frequency heteroplasmic SNPs and found hotspots linked to this pathology. Enhance-seq technology can be particularly useful here. Enhancers can be generated, and a panel of genes created to enhance very low-frequency SNPs in mitochondrial DNA.

### BRIEF DESCRIPTION OF THE FIGURES

FIG. 1—The primer used in mutation enhancement PCR. An Enhancer consists of 3 parts—the R-region or recognition region, the E-region or amplification region and a linker region. The R-region is designed to bind to the hotspot of the wild-type region of the gene. The first hybridization temperature enables this binding. In this case, the PCR cannot proceed due to the presence of a non-nucleotide structure in the linker region. B=If the hotspot region is mutated in the template gene, then binding is not efficient. C=If mutated, binding is more efficient with the E-region of the primer. In this case, PCR proceeds because it is not stopped by the non-nucleotide structure of the linker region.

FIG. 2: Enhancer design including a Bsal restriction site and amplification scheme for amplicon labelling.

FIG. 3: Illustration of the use of an oriented restriction site for the introduction of the Corrector sequences at the ends of amplicons.

FIG. 4: Principle of UMI addition by “A-tailing”: fate of a single molecule. Data analysis is performed using UMI clustering. To analyze the same mutations (here, L858R), the same DNA molecule will be encoded by different UMIs, so a higher sequencing depth is required due to multiple clustering (this technique corresponds to the “Safe-seq” method).

FIG. 5: Principle of UMI addition by A-tailing: becoming a single molecule. Strand specificity is given by a single base. This easily leads to sequencing errors (this technique is used in the “Duplex-Seq” method).

FIG. 6: Principle of UMI addition using the Golden gate method: becoming a single molecule. Each end of a DNA strand is uniquely marked and read in both directions, providing sequence information for the (+) and (−) strands. Each 4-base cohesive end is strand-specific, enabling asymmetrical mutations to be identified.

FIG. 7—This figure illustrates the method for inserting a restriction site (binding site) by PCR after enrichment PCR. A=primer pair containing a restriction site. B=representation of the amplicons obtained after PCR using the primer pair from step A. C=Golden gate binding of Corrector adapters to amplicons via restriction sites.

FIG. 8—This figure shows how the “Enhance-seq” method will be implemented if the initial sample is methylated DNA. A=the methylated DNA template will undergo bisulfide or enzymatic conversion or any other conversion method that will convert any unmethylated cytosine molecules into uracil molecules. Methylated cytosines, on the other hand, remain in cytosine form. The pattern of methylation protection is different in cancer and non-cancer patients. The methylation signature can be thought of as “mutations”. B=uracil-containing DNA is amplified by PCR to convert the uracil into a thymine molecule. C=The resulting DNA is ready to be used as template in the “Enhance-seq” method.

FIG. 9—This figure outlines the three main steps in the “Enhance-seq” method. The first two steps are part of the “Enhance-seq” molecular biology method, and the third step is software data correction. Step 1 is Mutation Enrichment PCR (MEP), which amplifies the mutation signal present in the initial population. In step 2, adapters or Corrector molecules are bound to the amplicons. PCR is performed to amplify the molecules so that each IMU present in the Corrector is replicated many times. In step 3, Corrector UMIs are used to correct sequencing errors in the amplicon. Each sequence from the MEP thus presents a UMI, and is amplified once again. A consensus is then drawn from all daughter sequences with the same UMI, enabling mutations to be distinguished from sequencing errors.

FIG. 10: Implementation of the method in multiplexing conditions coupled to a duplex-specific nuclease (DSN).

FIG. 11: Implementation of the method for mutant enrichment using marker coupled LNA bases.

FIG. 12: Mutation enrichment PCR analysis (on agarose gel). In lane 1, only the wild-type form and in lane 2, only the mutant form of the plasmids were used as templates. In lane 3, both the wild-type and the mutant in the ratio 98-2 were used. The expected PCR product is around 430 bp. In lane 1, no amplification is observed in the expected region. In tracks 2 and 3, on the other hand, strong amplification was observed.

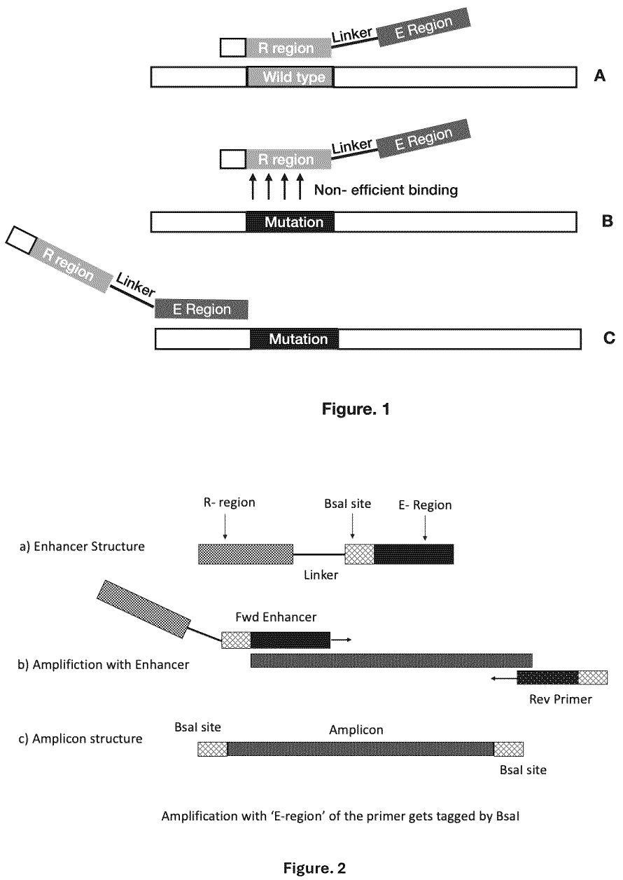

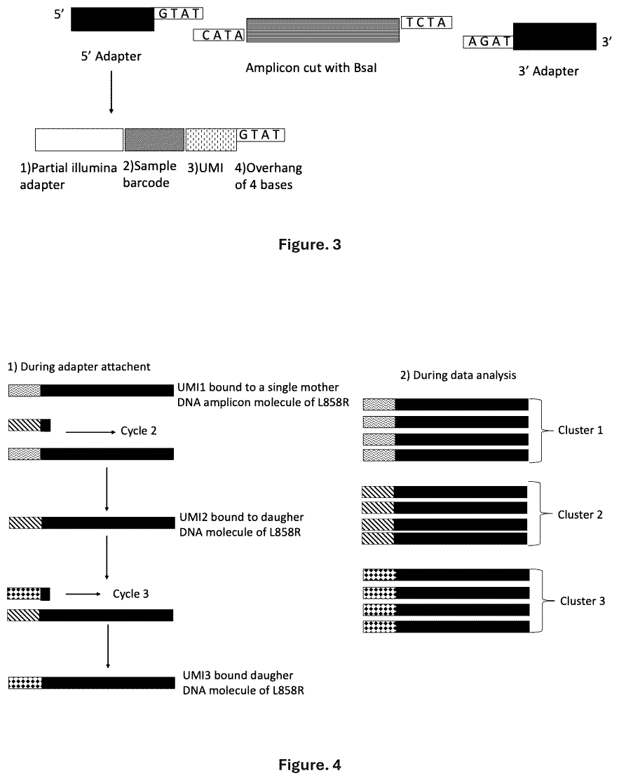

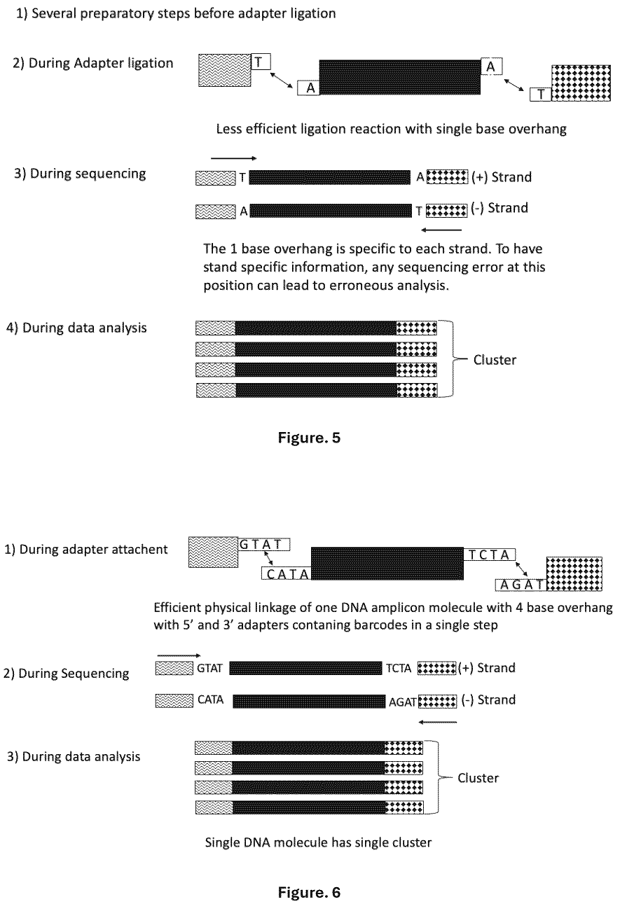

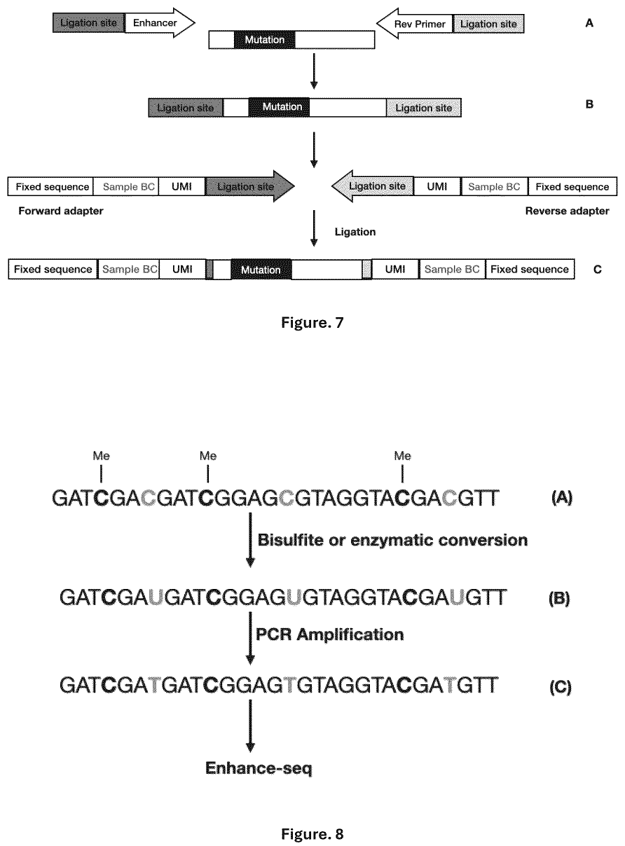

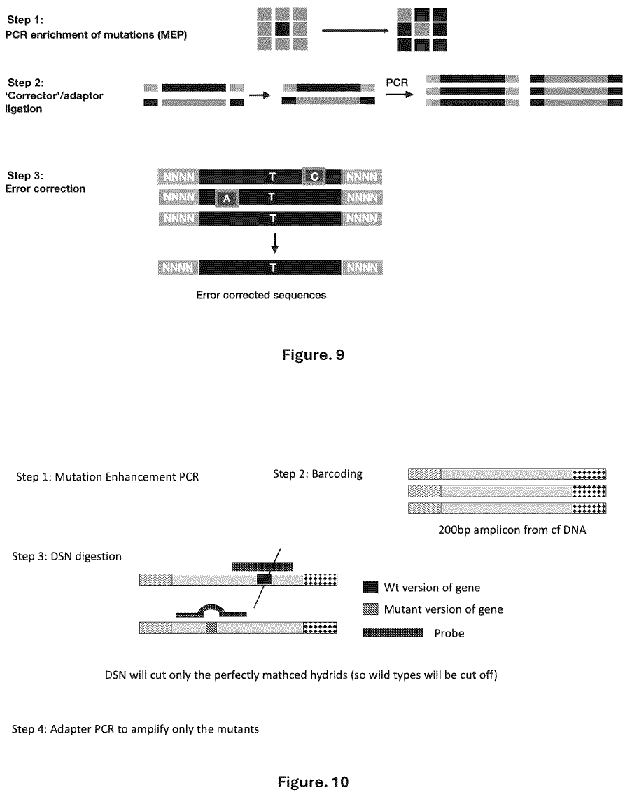

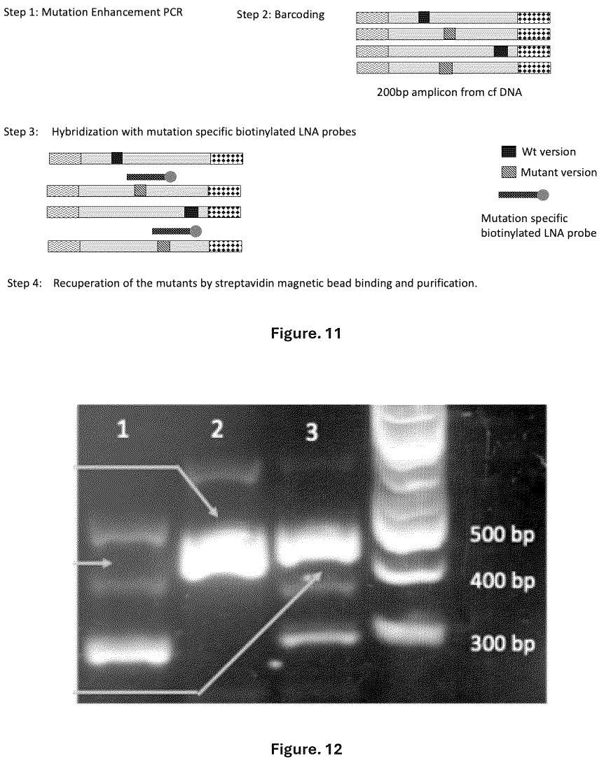

### EXPERIMENTAL PART

The following examples illustrate the reduction to practice of the method in the detection of a 15-base pair deletion on Exon 19 of the EGFR gene and of the point mutation (for example, EGFR L858R on Exon 21.

### I—Materials and Methods

1) Gene synthesis: Two different plasmids were constructed.
(i) wild-type versions of exons 19 and 21 of the human epidermal growth factor receptor gene (EGFR). Reported herein as pUC57-W.
(ii) mutated version of the exon 19 (15 bp deletion) and L858R of exon21 from the same EGFR gene, mentioned under the name pUC57-M.
   2) Mutation Enhancement PCR (MEP) with Enhancers:

Enhancers are specially designed primers to specifically enhance mutants, but not the wild type. The design of Enhancers is inspired by the article by Huang et al. 2019 which showed a new way to design primers (called “stuntmers”) to specifically enhance mutations in a context where wild-type DNA is very present. The first step of the Enhance-seq method is Mutation Amplification PCR (MEP), where mutated sequences are preferentially amplified over wild type using Enhancers or stuntmers. Following primer sequences were used in the experiments

### Exon 19 Deletion Mutation:

Fwd_Enhancer Exon_19
(SEQ ID NO. 1)
5′-CCCGTCGCTATCAAGGAATTAAGAGAAGCAAC-
C3-TAAAATTCCCGTCGCT-3′
Rev_Exon_19
(SEQ ID NO. 2)
5′-AGCAGCTGCCAGACATGAGA-3′

### Exon21 L858R Point Mutation:

Fwd_Enhancer Exon_21
(SEQ ID NO. 3)
5′-GATTTTGGGCTGGCCAAACTGCTGG-C3-
AGCATGTCAAGATCACAGATTTT-3′
Rev_Exon_21
(SEQ ID NO. 4)
5′-GAGCTCACCCAGAATGTCTGG-3′

Mutation Enhancement PCR (MEP) with Q5 Polymerase:

5× Master mix: 5 ul, dNTPs (10 mM): 0.5 ul, Fwd primer (10 mM): 1.25 ul, Rev primer (10 mM): 1.25 ul, template: variable, water: variable.

PCR conditions: Initial denaturation: 98° C. for 30 secs, denaturation: 98° C. for 10 secs, primary annealing: 70° C. for 30 secs, secondary annealing: 57° C. (for exon 19), and 59° C. (for exon 21) for 30 sec. extension: 72° C. for 30 secs. Final extension: 72° C. for 2 mins. For 45 cycles.

### 3) Addition of Ligation Site

In this experiment, binding sites were created by adding Bsal to each end of the amplicon.

Addition of the Bsal site by PCR with Q5 polymerase—5× buffer Q5-5 ul, dNTPs (10 mM)-0.5 ul, Fwd_barcoding_primer (10 mM)-1.25 ul, Rev_barcoding primer (10 mM)-1.25 ul, template-1 ul, water-10.5 ul.

PCR conditions-Initial denaturation −98° C. for 30 seconds, denaturation −98° C. for 10 seconds, hybridization-62° C. for 30 seconds, extension −72° C. for 30 seconds. Final extension −72° C. for 2 minutes. For 30 cycles

### 4) Double-Stranded DNA-Specific Nuclease (DSN) Treatment (Optional):

In the case where a duplex-specific nuclease (DSN) addition step is applied, the hybridization temperature of the Enhancers-R region is chosen to be around 67° C. The experiment proceeds as follows:
i) In a thermocycler, the temperature is raised to 98° C. to denature the DNA.
ii) The sample is cooled to 67° C. (first MEP hybridization temperature), DSN is added, and the reaction is incubated for 20 minutes.
iii) Incubation is followed by 2 minutes of DSN enzyme inactivation at 95° C.
iv) The sample is cooled again to the second MEP hybridization temperature and PCR is performed after addition of the necessary reagents.

### 5) End Preparation and Joining of Amplicons and Correctors:

Restriction digestion and binding of amplicons and Correctors are carried out in a single step using Golden gate technology.

T4 DNA ligase buffer-2.5 ul, Amplicon-2.3 ul, Fwd_corrector-0.56 ul, Rev_corrector-0.85 ul. Bsal-0.5 ul, T4 DNA ligase-0.5 ul, Water-16.8 ul.

Reactions are incubated at 37° C. for 40 minutes and inactivated at 60° C. for 10 minutes. The reactions are then cleaned up by PCR clean up kit and eluted in 10 ul elution buffer. Here, 6 bp X designates the sample or patient label; 10 bp N designates the degenerated UMI region.

Incubated at 37° C. for 40 mins and inactivated at 60 degrees for 10 mins

Cleaned up by PCR clean up kit and eluted in 10 ul of elution buffer.

### 6) Amplification of the Corrector Ligated Amplicons:

The amplicons ligated with correctors were amplified with a PCR using Q5 polymerase:

PCR reaction-5× Master mix: Sul; dNTPs (10 mM): 0.5 ul; Universal_Fwd_primer (10 mM): 1.25 ul; Universal_Rev_primer (10 mM): 1.25 ul; Template: 5 ul; Water: 12 ul.

PCR conditions: Initial denaturation −98° C. for 30 seconds, denaturation −98° C. for 10 seconds, annealing −65° C. for 30 seconds, extension −72° C. for 30 seconds. Final extension −72° C. for 2 minutes. For 30 cycles.

Universal_fwd_primer-
(SEQ ID NO. 5)
5′-ACACTCTTTCCCTACACGAC-3′
Universal_rev_primer-
(SEQ ID NO. 6)
5′-GACTGGAGTTCAGACGTGTG-3′

### 7) Spike-In Experiment

450-bp PCR products containing exon 19 and exon 21 were amplified from plasmids pUC57-W or M. They were then mixed at different concentrations. These amplicons were then added with human serum (Sigma-Aldrich) to mimic the clinical condition where cell-free DNA is mixed with human serum. Amplicons were then isolated from the serum.

8) Isolation of Amplicons from Human Serum

Applied Biosystem's MagMAX™ Cell-Free DNA Isolation Kit was used to isolate amplicons from the serum sample. This kit is designed to isolate circulating DNA from cell-free human plasma, serum and urine samples, using Dynabeads magnetic beads. Manufacturer's instructions were followed to isolate amplicons from serum. Isolated amplicons were eluted in 20 ul of elution buffer.

### Results

### Example 1—Agarose Gel Analysis of Mutation Enrichment PCR

Mutation enrichment PCRs (MEPs) were performed for the Exon19 deletion mutation with the primers Fwd_exon19_Enhancer and Reverse_exon21 using different plasmid templates-wild-type pUC57-W, mutant pUC57-M and mixtures of these two in ratios (98-2) to see if the effect of mutation-enriching PCR can be observed visually in the agarose gel. All templates were used at concentrations of 10 ng/ul. After 35 cycles of MEP, the amplicons were loaded into the 2% agarose gel. The expected PCR product is around 430 bp for the wild-type sequence and 415 bp for the mutant form. No amplification was observed in the case of the wild type; in contrast, strong amplification bands were observed when a mutant or a mixture of wild-type and mutants was used as template (FIG. 12).

### Example 2—Trials with Different Ratios of Wild-Type and Mutant Populations

The aim of this experiment was to quantify the increase in the mutation signal and to see whether 30 several PCR cycles are required to significantly increase the mutant population. To this end, wild-type pUC57-W and mutant pUC57-M plasmids were diluted to a concentration of 10 ng/ul each and then used in two different ratios. Mixture of mutant and wild-type in a ratio of 0.2:99.8 (R1).

Mixture of mutant and wildtype in a ratio of 2:98 (R2).

Mutation enrichment PCRs were performed using these two mixtures as initial templates. Three consecutive rounds of PCR were performed using the amplicon from the previous round as the template for the subsequent PCR. Each time, after one PCR cycle, the product was purified and diluted to a concentration of 10 ng/ul before proceeding to the next PCR step. The results show that a single round of PCR (here 35 cycles) is sufficient to saturate the mutation and block wild-type amplification, and that several additional rounds of PCR do not increase the number of mutants in the population.

For exon 19 deletion mutation—

TABLE 3
Change in wild-type and mutant ratios after each PCR cycle.

| Initial template ratio of | | Ratio of Wt:Mutant | Fold increase |
|---|---|---|---|
| Wt:Mutant | PCR | after PCR | for mutant |
| 98:2 (R1) | 1^{st} PCR | 0.06:99.94 | 50 |
| 0.06:99.94 | 2^{nd} PCR | 0.18:99.81 | — |
| 0.18:99.81 | 3^{rd} PCR | 0.3:99.7 | — |
| 99.8:0.2 (R2) | 1^{st} PCR | 0.06:99.94 | ~500 |
| 0.67:99.33 | 2^{nd} PCR | 0.49:99.51 | — |

For Exon21 point mutation L858R—

TABLE 4
Change in wild-type and mutant ratios after each PCR cycle.

| Initial template ratio | | Final template ratio | Fold increase for |
|---|---|---|---|
| (WT:Mutant) | PCR | (WT:Mutant) | mutant |
| 98:2 (R1) | 1st PCR | 49:51 | 25 |
| 49:51 | 2nd PCR | 19:81 | — |

### Example 3—Template Concentrations in Absolute Numbers

This experiment aims to identify the detection threshold of MEP and traditional PCR in absolute numbers. For this purpose, pUC57-W and pUC57-M were diluted to obtain the following concentrations.

Table 3. Change in wild-type and mutant ratios after each PCR cycle. For Exon21 point mutation L858R per 10 ul-1) 10 molecules of mutant plasmid and 10\hat{ }8 molecules of wild-type plasmid (0.00001%) 2) 10 molecules of mutant plasmid and 10\hat{ }9 molecules of wild-type plasmid (0.000001%)

Conventional PCR (with a single hybridization temperature) and mutation enrichment PCR (MEP) for Exon19 deletion mutation and Exon 21 point mutation L858R were performed using these templates. The amplicons were sequenced by NGS sequencing and data analysis revealed the following results in Table 5.

TABLE 5
Effect of traditional PCR and MEP PCR on the absolute
number of mutations present after amplification.

| | | | Ratio of | |
|---|---|---|---|---|
| | Wt | | Wt:Mutant | Results |
| EGFR genes | molecule/sample | PCR | after NGS | (NGS) |
| Exon 19 | 10\hat{ }8 | MEP | 52:48 | 57 times |
| Exon 19 | 10\hat{ }9 | MEP | 52:48 | 54 times |
| Exon 21 | 10\hat{ }8 | MEP | 91:9 | 64 times |
| Exon 21 | 10\hat{ }9 | MEP | 98.6:1.3 | 16 times |
| Exon 19 | 10\hat{ }8 | Traditional | 99.16:0.84 | — |
| Exon 19 | 10\hat{ }9 | Traditional | 99.11:0.89 | — |
| Exon 21 | 10\hat{ }8 | Traditional | 99.86:0.14 | — |
| Exon 21 | 10\hat{ }9 | Traditional | 99.92:0.08 | — |

Experimental data show that in MEP the deletion mutation in exon 19 is detected at least 5 times more efficiently than the L858R point mutation in exon 21. MEP can detect mutation at least 10 to 60 times more efficiently in hyper-diluted concentrations of mutant DNA, where 10 molecules of mutant DNA or less (according to Poisson statistics) are present in the background of 100 million or 1 billion wild-type DNA sequences. In the ratio of 10 to 1 billion mutant DNA molecules to wild-type DNA, MEP has shown amplification capacities 10 to 50 times higher, depending on the nature of the mutation (point mutation or deletion). This will enable extremely low quantities of mutations present in the population to be detected in shallow sequencing machines, saving on investment.

### Example 4—Spike-In Experiment with Amplicons in Serum

The aim of this experiment was to see the performance of the Enhance-seq method on serum samples with added artificial genes. In a complex solution like serum, the isolation of free circulating DNA (cfDNA) could be a bottleneck to reach the same detection/sensitivity limit as in water, as previously done in the synthetic experiment.

pUC57-W and pUC57-W were amplified to obtain two different 450-base-pair amplicons. These wild-type and mutant amplicon DNAs were diluted to a concentration of 2 ng/ml and then mixed in different ratios. The amplicons were then added to 500 ul of serum at the following concentrations.

TABLE 6
Preparation of test samples

| Sample | Ratio between Wt:Mutant | Volume mixed in 500 ul of serum | |
|---|---|---|---|
| 1 | 10 mutants:10\hat{ }7 Wt/10 ul | 10 | ul |
| 2 | 10 mutants:10\hat{ }7 Wt/10 ul | 50 | ul |
| 3 | 10 mutants:10\hat{ }7 Wt/10 ul | 100 | ul |

Amplicons were then extracted from serum using the cfDNA MegaMax isolation kit and eluted in 20 ul of elution buffer. Traditional PCR or MEP was performed for the L858R point mutation in exon 21 with 10 ul of template. MEP was performed for exon 21 with the following components—5 5× Q5 Buffer-5 ul, dNTPs-0.5 ul, Fwd_Enhancer Exon_21-1.25 ul, 21 Reverse P-1.25 ul, Q5 polymerase—0.25 ul, template DNA-5 ul, Water-12 ul. MEP conditions

98° C. for 30 seconds, denaturation −98° C. for 10 seconds, primary hybridization −70° C. for 20 seconds, secondary hybridization −59° C. for 20 seconds. Extension −72° C. for 30 seconds. Final extension −72° C. for 2 minutes. For 45 cycles.

TABLE 7
Effect of conventional PCR and MEP on hyper-diluted
mutated amplicons isolated from serum.

| | Wt molecules in | Mutant molecules | | Type of | Ratio Wt:Mut |
|---|---|---|---|---|---|
| Sample | template | in templete | | PCR | after NGS |
| M5 MEP | 5 × 10\hat{ }6 | ~5 | molecules | MEP | 2.8:97.2 |
| M50 MEP | 2.5 × 10\hat{ }7 | ~50 | molecules | MEP | 2.8:97.4 |
| M500 MEP | 5 × 10\hat{ }7 | ~500 | molecules | MEP | 7:93 |
| M5 Trad | 5 × 10\hat{ }6 | ~5 | molecules | Traditional | 78:22 |
| M50 Trad | 2.5 × 10\hat{ }7 | ~50 | molecules | Traditional | 79:21 |
| M500 Trad | 5 × 10\hat{ }7 | ~500 | molecules | Traditional | 77:23 |

This result shows that MEP can amplify mutant DNA around five times more efficiently than conventional PCR using a single hybridization temperature and conventional primer design. The system's efficiency is higher when using a linear DNA fragment than in plasmid form.

### Example 5—Detection of KRAS Mutants (KRAS G12D, G13S, Q22K, R68S, a 146T) at 0.001% VAF by Multiplexing Materials and Methods

### 1. Gene Synthesis

Two plasmids, PUC57 containing either the wild-type KRAS gene or the mutant KRAS gene, were constructed. They were mixed so that the ratio of mutant to wild-type plasmid was 0.001%.

2. Mutation Enhancement PCR (MEP) with Enhancers

Enhancers were designed to amplify mutant DNA sequences preferentially to wild-type sequences. Enhancers containing LNA (locked nucleic acid) bases at the mutation site in the R region have been designed. They can easily differentiate between wild-type and mutant bases. One of the advantages of using LNA is the increased melting temperature of the LNA: DNA pair in the wild-type template compared with the mutant template, enabling more efficient discrimination and amplification of the mutant template. Primer sequences are listed below. Locked nucleic acids are indicated in square brackets [ ].

KRAS G12D G13S Q22K
KRAS1 Fwd:
(SEQ ID NO. 7)
5′-GGAGCT[G]GTG[G]CGTAGGCAAGAG-C3-
ATGATTGGTCTCAGTATTGTGGTAGTTGGAGCT-3′
KRAS1 Rev:
(SEQ ID NO. 8)
5′-ATCAAAGAATGGTCCTGCACC
KRAS A146T
KRAS2 Fwd:
(SEQ ID NO. 9)
5′-AACAT[CAG]CAAAGACAAGACAGGTAAGTAAC
AC-C3-ATGATTGGTCTCAGTATGGAATTCCTTTTAT
TGAAACATC-3′
KRAS2 Rev:
(SEQ ID NO. 10)
5′-GACATAACAGTTATGATTTTGCAGAAAACAG-3′
KRAS3 R68S
KRAS3 Fwd:
(SEQ ID NO. 11)
5′-CAATGAG[G]GACCAGTACATGAGGACTGGGGA-
C3-ATGATTGGTCTCAGTATGGAGTACAGTGCAATGA3′
KRAS3-Rev:
(SEQ ID NO. 12)
5′-CCAATTTAAACCCACCTATAATGGTGAATATCTTC-3′

### 3. Enrichment PCR

Q5 Master mix 2×: 25 ul, Primer mix (100 nM): 3 ul Template: variable, water: variable. Conditions de la PCR: dénaturation initial: 98° C. for 30 secondes; denaturation: 98° C. for 10 secondes; first hybridation: 76° C. for 60 secondes; second hybridation: 57° C. for 30 secondes. Extension: 72° C. for 30 secondes. Extension finale: 72° C. for 2 minutes. For 45 cycles

### 4. End Preparation and Ligation of Amplicons and Correctors

Amplicon and Corrector restriction site digestion and binding are performed in a single step using Golden gate technology. T4 DNA ligase buffer: 2.5 ul, Amplicon: 20 ng, Fwd_barcode: 10 ng, Rev_barcode: 10 ng Golden gate (GG) enzyme mix: 1 ul, Water-until 25 ul. Reactions are incubated at 37° C. for 1 h and inactivated at 60° C. for 10 mins. Reaction products are then purified using the PCR purification kit and eluted in 20 ul elution buffer.

Fwd_corrector:
(SEQ ID NO. 13)
5′ACACTCTTTCCCTACACGACGCTCTTCCGATCTXXXX
XXNNNNNNNNNNGTATAGAGACCATTGTAGTAGCG-3′
Rev_corrector:
(SEQ ID NO. 14)
5′GACTGGAGTTCAGACGTGTGCTCTTCCGATCTXXX
XXXNNNNNNNNNNAGATGGAGACCTTGATGTAGC-3′

Here, XXXXXX signifies the sample or patient barcode; NNNNNNNNNNN designates the degenerate UMI region.

### 5. Amplification of Amplicons and Adding Illumina Adapters

Amplicons containing Correctors were amplified with PCR using Q5's 2×master mix:

PCR reaction-2× Master mix: 25 ul; 0.5 ul, P5_fwd_primer (500 nM): 1 ul; P7 reverse primer (500 nM): 1 ul; Template: 50 ng; Water: 12 ul.

PCR conditions: Initial denaturation: 98° C. for 30 seconds, denaturation: 98° C. for 10 seconds, annealing: 65° C. for 30 seconds, extension: 72° C. for 30 seconds. Final extension: 72° C. for 2 minutes. For 10 cycles.

P5_fwd_primer:
(SEQ ID NO.15)
5′-AATGATACGGCGACCACCGAGATCTACACTCT
TTCCCTACACGAC-3′
P7_rev_primer:
(SEQ ID NO. 16)
5′-CAAGCAGAAGACGGCATACGAGATGTGACTGG
AGTTCAGACGTGTG-3′

### Results:

TABLE 8
Change in mutant gene ratios before and after MEP. The negative
control containing 100% WT and no mutant shows no mutant
DNA after MEP, suggesting the absence of contamination in
our procedure. VAF is the mutant/wild-type ratio.

| | Initial | Final | Mutant |
|---|---|---|---|
| Gene | VAF | VAF | augmentation |
| KRAS G12D G13S Q22K | 0.001% | 15% | x 15000 |
| KRAS R68S | 0.001% | 6% | X 6000 |
| KRAS A146T | 0.001% | 3% | x 3000 |
| KRAS WT (control) | 0% | 0% | NA |

### REFERENCES

1. Abbosh, C., Birkbak, N. J., Wilson, G. A., Jamal-Hanjani, M., Constantin, T., Salari, R., . . . & Swanton, C. (2017). Phylogenetic ctDNA analysis depicts early-stage lung cancer evolution. Nature, 545(7655), 446-451.
2. Atilano, S. R., Udar, N., Satalich, T. A., Udar, V., Chwa, M., & Kenney, M. C. (2021). Low frequency mitochondrial DNA heteroplasmy SNPs in blood, retina, and [RPE+choroid] of age-related macular degeneration subjects. Plos one, 16(1), e0246114.
3. Chabon, J. J., Simmons, A. D., Lovejoy, A. F., Esfahani, M. S., Newman, A. M., Haringsma, H. J., . . . & Diehn, M. (2016). Circulating tumour DNA profiling reveals heterogeneity of EGFR inhibitor resistance mechanisms in lung cancer patients. Nature communications, 7(1), 1-15.
4. Chatterton, Z., Mendelev, N., Chen, S., Raj, T., Walker, R., Carr, W., . . . & Haghighi, F. (2019). Brain-derived circulating cell-free DNA defines the brain region and cell specific origins associated with neuronal atrophy. bioRxiv, 538827.
5. Cheng, A. P., Cheng, M. P., Loy, C. J., Lenz, J. S., Chen, K., Burnham P., Timblin M., Orejas J L., Silverman E., Marty F M., Ritz J, and De Vlaminck, I. (2020). BioRXiV. Cell-free DNA Profiling Informs Major Complications of Hematopoietic Cell Transplantation. bioRxiv, 2020-04.
6. Couraud, S., Vaca-Paniagua, F., Villar, S., Oliver, J., Schuster, T., Blanche, H., . . . & Souquet, P. J. (2014). Noninvasive diagnosis of actionable mutations by deep sequencing of circulating free DNA in lung cancer from never-smokers-a proof-of-concept study from BioCAST/IFCT-1002. Clinical Cancer Research, 20(17), 4613-4624.
7. De Fraipont, F., Gazzeri, S., Cho, W. C., & Eymin, B. (2019). Circular RNAs and RNA splice variants as biomarkers for prognosis and therapeutic response in the liquid biopsies of lung cancer patients. Frontiers in genetics, 10, 390.
8. Diehn, M., Alizadeh, A., Newman, A., Klass, D. M. Identification and use of circulating Nucleic acids. WO 2016/040901 A1.
9. González-Billalabeitia, E., Conteduca, V., Wetterskog, D., Jayaram, A., & Attard, G. (2019). Circulating tumor DNA in advanced prostate cancer-transitioning from discovery to a clinically implemented test. Prostate cancer and prostatic diseases, 22(2), 195-205.
10. Ferrero, L., Cameron, B., & Crouzet, J. (1995). Analysis of gyrA and grlA mutations in stepwise-selected ciprofloxacin-resistant mutants of Staphylococcus aureus. Antimicrobial Agents and Chemotherapy, 39(7), 1554-1558.
11. Fiala, C., & Diamandis, E. P. (2018). Utility of circulating tumor DNA in cancer diagnostics with emphasis on early detection. BMC medicine, 16(1), 1-10.
12. Flandrois, J. P., Lina, G., & Dumitrescu, O. (2014). MUBII-TB-DB-a database of mutations associated with antibiotic resistance in Mycobacterium tuberculosis. BMC bioinformatics, 15(1), 1-9.
13. Forshew, T., Murtaza, M., Parkinson, C., Gale, D., Tsui, D. W., Kaper, F., . . . & Rosenfeld, N. (2012). Noninvasive identification and monitoring of cancer mutations by targeted deep sequencing of plasma DNA. Science translational medicine, 4(136), 136ra68-136ra68.
14. Fredebohm, J., Mehnert, D. H., Löber, A. K., Holtrup, F., van Rahden, V., Angenendt, P., & Diehl, F. (2016). Detection and quantification of KIT mutations in ctDNA by plasma safe-seqS. In Circulating Nucleic Acids in Serum and Plasma-CNAPS IX (pp. 187-189). Springer, Cham.
15. Gale, D., Lawson, A. R., Howarth, K., Madi, M., Durham, B., Smalley, S., . . . & Rosenfeld, N. (2018). Development of a highly sensitive liquid biopsy platform to detect clinically-relevant cancer mutations at low allele fractions in cell-free DNA. PloS one, 13(3), e0194630.
16. Glenn, T. C. (2011). Field guide to next-generation DNA sequencers. Molecular ecology resources, 11(5), 759-769.
17. González-Billalabeitia, E., Conteduca, V., Wetterskog, D., Jayaram, A., & Attard, G. (2019). Circulating tumor DNA in advanced prostate cancer-transitioning from discovery to a clinically implemented test. Prostate cancer and prostatic diseases, 22(2), 195-205.
18. How-Kit, A., Mazaleyrat, N., Daunay, A., Nielsen, H. M., Terris, B., & Tost, J. (2013). Sensitive Detection of KRAS Mutations Using Enhanced-ice-COLD-PCR Mutation Enrichment and Direct Sequence Identification. Human mutation, 34(11), 1568-1580.
19. Huang, J. K., Fan, L., Wang, T. Y., & Wu, P. S. (2019). A new primer construction technique that effectively increases amplification of rare mutant templates in samples. BMC biotechnology, 19(1), 1-11.
20. Ilié, M., & Hofman, P. (2016). Pros-Can tissue biopsy be replaced by liquid biopsy?. Translational lung cancer research, 5(4), 420.
21. Kennedy, S. R., Schmitt, M. W., Fox, E. J., Kohrn, B. F., Salk, J. J., Ahn, E. H., . . . & Loeb, L. A. (2014). Detecting ultralow-frequency mutations by Duplex Sequencing. Nature protocols, 9(11), 2586-2606.
22. Keraite, I., Alvarez-Garcia, V., Garcia-Murillas, I., Beaney, M., Turner, N. C., Bartos, C., . . . & Leslie, N. R. (2020). PIK3CA mutation enrichment and quantitation from blood and tissue. Scientific reports, 10(1), 1-12.
23. Khush, K. K., Patel, J., Pinney, S., Kao, A., Alharethi, R., DePasquale, E., . . . & Kobashigawa, J. (2019). Noninvasive detection of graft injury after heart transplant using donor-derived cell-free DNA-a prospective multicenter study. American Journal of Transplantation, 19(10), 2889-2899.
24. Kinde, I., Wu, J., Papadopoulos, N., Kinzler, K. W., & Vogelstein, B. (2011). Detection and quantification of rare mutations with massively parallel sequencing. Proceedings of the National Academy of Sciences, 108(23), 9530-9535.
25. Li, J., Wang, L., Mamon, H., Kulke, M. H., Berbeco, R., & Makrigiorgos, G. M. (2008). Replacing PCR with COLD-PCR enriches variant DNA sequences and redefines the sensitivity of genetic testing. Nature medicine, 14(5), 579-584.
26. Li J, Makrigiorgos GM. COLD-PCR-a new platform for highly improved mutation detection in cancer and genetic testing. Biochem Soc Trans. 2009; 37 (Pt 2)-427-32.
27. Martuszewski, A., Paluszkiewicz, P., Król, M., Banasik, M., & Kepinska, M. (2021). Donor-Derived Cell-Free DNA in Kidney Transplantation as a Potential Rejection Biomarker-A Systematic Literature Review. Journal of Clinical Medicine, 10(2), 193.
28. Melnyk, A. H., Wong, A., & Kassen, R. (2015). The fitness costs of antibiotic resistance mutations. Evolutionary applications, 8(3), 273-283.
29. Milbury, C. A., Li, J., & Makrigiorgos, G. M. (2011). Ice-COLD-PCR enables rapid amplification and robust enrichment for low-abundance unknown DNA mutations. Nucleic acids research, 39(1), e2-e2.
30. Mirkov, M. U., Verstockt, B., & Cleynen, I. (2017). Genetics of inflammatory bowel disease-beyond NOD2. The lancet Gastroenterology & hepatology, 2(3), 224-234.
31. Narayan, A., Carriero, N. J., Gettinger, S. N., Kluytenaar, J., Kozak, K. R., Yock, T. I., . . . & Patel, A. A. (2012). Ultrasensitive measurement of hotspot mutations in tumor DNA in blood using error-suppressed multiplexed deep sequencing. Cancer research, 72(14), 3492-3498.
32. Newman, A. M., Bratman, S. V., To, J., Wynne, J. F., Eclov, N. C., Modlin, L. A., . . . & Diehn, M. (2014). An ultrasensitive method for quantitating circulating tumor DNA with broad patient coverage. Nature medicine, 20(5), 548-554.
33. Newman, A. M., Lovejoy, A. F., Klass, D. M., Kurtz, D. M., Chabon, J. J., Scherer, F., . . . & Alizadeh, A. A. (2016). Integrated digital error suppression for improved detection of circulating tumor DNA. Nature biotechnology, 34(5), 547-555.
34. Nicolas, G., Acuña-Hidalgo, R., Keogh, M. J., Quenez, O., Steehouwer, M., Lelieveld, S., . . . & Hoischen, A. (2018). Somatic variants in autosomal dominant genes are a rare cause of sporadic Alzheimer's disease. Alzheimer's & Dementia, 14(12), 1632-1639.
35. Nilsson, R. J. A., Karachaliou, N., Berenguer, J., Gimenez-Capitan, A., Schellen, P., Teixido, C., . . . & Wurdinger, T. (2016). Rearranged EMLA-ALK fusion transcripts sequester in circulating blood platelets and enable blood-based crizotinib response monitoring in non-small-cell lung cancer. Oncotarget, 7(1), 1066.
36. Olafsson, S., McIntyre, R. E., Coorens, T., Butler, T., Jung, H., Robinson, P. S., . . . & Anderson, C. A. (2020). Somatic evolution in non-neoplastic IBD-affected colon. Cell, 182(3), 672-684.
37. Poole, J. C., Wu, S. F., Lu, T. T., Vibat, C. R. T., Pham, A., Samuelsz, E., . . . & Arnold, L. J. (2019). Analytical validation of the Target Selector ctDNA platform featuring single copy detection sensitivity for clinically actionable EGFR, BRAF, and KRAS mutations. PloS one, 14(10), e0223112.
38. Qin, Z., Ljubimov, V. A., Zhou, C., Tong, Y., & Liang, J. (2016). Cell-free circulating tumor DNA in cancer. Chinese journal of cancer, 35(1), 1-9.
39. Ross, K. A. (2014). Coherent somatic mutation in autoimmune disease. PloS one, 9(7), e101093.
40. Savola, P., Kelkka, T., Rajala, H. L., Kuuliala, A., Kuuliala, K., Eldfors, S., . . . & Mustjoki, S. (2017). Somatic mutations in clonally expanded cytotoxic T lymphocytes in patients with newly diagnosed rheumatoid arthritis. Nature communications, 8(1), 1-14.
41. Schmitt, M. W., Kennedy, S. R., Salk, J. J., Fox, E. J., Hiatt, J. B., & Loeb, L. A. (2012). Detection of ultra-rare mutations by next-generation sequencing. Proceedings of the National Academy of Sciences, 109(36), 14508-14513.
42. Schwarzenbach, H., Stoehlmacher, J., Pantel, K., & Goekkurt, E. (2008). Detection and monitoring of cell-free DNA in blood of patients with colorectal cancer. Annals of the New York Academy of Sciences, 1137(1), 190-196.
43. Schultz, M. D., He, Y., Whitaker, J. W., Hariharan, M., Mukamel, E. A., Leung, D., . . . & Ecker, J. R. (2015). Human maps body epigenome reveal noncanonical DNA methylation variation. Nature, 523(7559), 212-216.
44. Shagin, D. A., Rebrikov, D. V., Kozhemyako, V. B., Altshuler, I. M., Shcheglov, A. S., Zhulidov, P. A., . . . & Lukyanov, S. (2002). A novel method for SNP detection using a new duplex-specific nuclease from crab hepatopancreas. Genome research, 12(12), 1935-1942.
45. Song, C., Liu, Y., Fontana, R., Makrigiorgos, A., Mamon, H., Kulke, M. H., & Makrigiorgos, G. M. (2016). Elimination of unaltered DNA in mixed clinical samples via nuclease-assisted minor-allele enrichment. Nucleic acids research, 44(19), e146-e146.
46. Stroun, M., Anker, P., Lyautey, J., Lederrey, C., & Maurice, P. A. (1987). Isolation and characterization of DNA from the plasma of cancer patients. European Journal of Cancer and Clinical Oncology, 23(6), 707-712.
47. Tie, J., Kinde, I., Wang, Y., Wong, H. L., Roebert, J., Christie, M., . . . & Gibbs, P. (2015). Circulating tumor DNA as an early marker of therapeutic response in patients with metastatic colorectal cancer. Annals of Oncology, 26(8), 1715-1722.
48. Tie, J., Wang, Y., Tomasetti, C., Li, L., Springer, S., Kinde, I., . . . & Gibbs, P. (2016). Circulating tumor DNA analysis detects minimal residual disease and predicts recurrence in patients with stage II colon cancer. Science translational medicine, 8(346), 346ra92-346ra92.
49. Uchida, J., Kato, K., Kukita, Y., Kumagai, T., Nishino, K., Daga, H., . . . & Imamura, F. (2015). Diagnostic accuracy of noninvasive genotyping of EGFR in lung cancer patients by deep sequencing of plasma cell-free DNA. Clinical chemistry, 61(9), 1191-1196.

1-14. (canceled)

15. A method for the detection of a rare mutation in a biological sample, comprising the steps of:
Step 1: Providing a sample comprising DNA
Step 2: Amplifying selectively the region containing said mutation using Mutation Enhancement PCR (MEP) technology, which includes the following steps:
a) Providing a PCR primer (Enhancer) comprising 3 regions
(i) a region called “R-region”, which specifically binds to the wild-type sequence but cannot bind if there is a mutation.
(ii) a linker region and
(iii) a region called “E-region”, which specifically binds to a sequence located ahead (downstream) of the mutation site;
b) Amplification of the template DNA by a PCR comprising two hybridization steps: (i) a first hybridization at a temperature T1 which allows binding of the R-region of the primer to the wild-type sequence and (ii) a second hybridization at a temperature T2 which allows binding of the E-region of the primer; wherein T1 is at least 5° C. higher than T2;
c) In case of binding of the E-region, the sequence containing the said mutation is amplified
d) Obtaining amplicons containing the said mutation. The said amplicons containing an oriented restriction enzyme site, the said oriented restriction enzyme site being added either during the MEP, or during an additional PCR after step c) of the said MEP, using primers containing an oriented restriction enzyme site;
Step 3: Adding Corrector sequence at each end of the resulting amplicon by PCR, comprising a fixed sequence, a unique molecular identifier (UMI), an oriented restriction enzyme site compatible with the oriented restriction enzyme sites presents in the extremities of the said amplicons, and optionally a barcode specific to the sample/patient, by using the said restriction sites;
Step 4: Amplifying amplicons resulting from Step 3 by PCR;
Step 5: Sequencing amplicons resulting from step 4 by NGS sequencing; and
Step 6: Correcting sequencing errors by bioinformatic analysis using the unique molecular identifiers.

16. The method according to claim 15, wherein a nuclease specific to the double-stranded DNA is added, either before PCR enrichment of the mutation, or after PCR enrichment of the mutation.

17. The method according to claim 15, wherein said amplifying primer comprises LNA bases either in the-R region.

18. The method according to claim 15, wherein barcodes are added either at the time of enrichment PCR due to the presence of such barcodes in the E-region, or at the same time as said oriented restriction site when the latter is added by PCR, or at step 3 due to its presence in the Corrector sequence.

19. The method according to claim 15, wherein the said rare mutation can be a point mutation, a deletion, an insertion or a rearrangement.

20. The method according to claim 15, wherein the rare mutation corresponds to the presence of a cytosine as a marker of DNA methylation, said method comprising a step of treatment of the DNA sample before DNA amplification of Step 2 in order to reveal the methylation site.

21. The method according to claim 15, said method comprising a step of treatment of the biological sample before DNA amplification of Step 2 in order to convert RNA into DNA.

22. The method according to claim 15, wherein a Patient barcode or Sample barcode is present in the Corrector sequence at Step 3 and then amplicons resulting from Step 4 are pooled together for NGS sequencing.

23. The method according to claim 15, wherein the biological sample is a liquid biopsy.

24. The method according to claim 15, wherein the method comprises the addition, after the PCR enrichment of the mutation and the addition of the Corrector sequences, of probes specific to wild-type sequences comprising LNA bases as well as a duplex-specific nuclease.

25. The method according to claim 15, wherein the method comprises the addition of mutation-specific probes comprising LNA base(s) coupled to a marker after the amplification of step 2 and the addition of Corrector sequences of step 3.

26. An amplifying primer comprising 3 regions:
a. the R region which recognizes and binds specifically to the wild-type sequence but cannot bind if the region contains a mutation,
b. a linker region and
c. the E region, which binds specifically to a sequence located upstream of the mutation site and comprises the sequence of an oriented restriction site positioned upstream of the E-region.

27. The amplifying primer according to claim 26, wherein said restriction site is Bsal.

28. The amplifying primer according to claim 26, further comprising LNA bases in the R-region.


SEQUENCE LISTING (16 sequences)

SEQ ID NO: 1 - DNA, 48 bp, synthetic construct
  source 1..48: mol_type = other DNA; note = c3 spacer
  misc_feature 32^33
cccgtcgctatcaaggaattaagagaagcaactaaaattcccgtcgct

SEQ ID NO: 2 - DNA, 20 bp, synthetic construct
  source 1..20: mol_type = other DNA
agcagctgccagacatgaga

SEQ ID NO: 3 - DNA, 47 bp, synthetic construct
  source 1..47: mol_type = other DNA
  misc_feature 24^25: note = c3 spacer
gattttggctggccaaactgctggagcatgtcaagatcacagatttt

SEQ ID NO: 4 - DNA, 21 bp, synthetic construct
  source 1..21: mol_type = other DNA
gagctcacccagaatgtctgg

SEQ ID NO: 5 - DNA, 20 bp, synthetic construct
  source 1..20: mol_type = other DNA
acactctttccctacacgac

SEQ ID NO: 6 - DNA, 20 bp, synthetic construct
  source 1..20: mol_type = other DNA
gactggagttcagacgtgtg

SEQ ID NO: 7 - DNA, 56 bp, synthetic construct
  source 1..56: mol_type = other DNA
  misc_feature 23^24: note = c3 spacer
ggagctggtggcgtaggcaagagatgattggtctcagtattgtggtagttggagct

SEQ ID NO: 8 - DNA, 21 bp, synthetic construct
  source 1..21: mol_type = other DNA
atcaaagaatggtcctgcacc

SEQ ID NO: 9 - DNA, 74 bp, synthetic construct
  source 1..74: mol_type = other DNA
  misc_feature 33^34: note = c3 spacer
aacatcagcaaagacaagacaggtaagtaacaccatgattggtctcagtatggaattccttttattgaaacatc

SEQ ID NO: 10 - DNA, 31 bp, synthetic construct
  source 1..31: mol_type = other DNA
gacataacagttatgattttgcagaaaacag

SEQ ID NO: 11 - DNA, 65 bp, synthetic construct
  source 1..65: mol_type = other DNA
  misc_feature 31^32: note = c3 spacer
caatgagggaccagtacatgaggactggggaatgattggtctcagtatggagtacagtgcaatga

SEQ ID NO: 12 - DNA, 35 bp, synthetic construct
  source 1..35: mol_type = other DNA
ccaatttaaacccacctataatggtgaatatcttc

SEQ ID NO: 13 - DNA, 56 bp, synthetic construct
  source 1..56: mol_type = other DNA
  misc_feature 33^34: note = barecode followed by UMI
acactctttccctacacgacgctcttccgatctgtatagagaccattgtagtagcg

SEQ ID NO: 14 - DNA, 53 bp, synthetic construct
  source 1..53: mol_type = other DNA
  misc_feature 32^33: note = barecode followed by UMI
gactggagttcagacgtgtgctcttccgatctagatggagaccttgatgtagc

SEQ ID NO: 15 - DNA, 45 bp, synthetic construct
  source 1..45: mol_type = other DNA
aatgatacggcgaccaccgagatctacactctttccctacacgac

SEQ ID NO: 16 - DNA, 46 bp, synthetic construct
  source 1..46: mol_type = other DNA
caagcagaagacggcatacgagatgtgactggagttcagacgtgtg